(19) United States
(12) Patent Application Publication
(10) Pub. No.: US 2016/0317755 A1
(43) Pub. Date: Nov. 3, 2016

(54) SAFETY SYRINGE

(71) Applicants: Hsien-Tsung WANG, (US); Wan-Hwa CHENG, (US)

(72) Inventors: Hsien-Tsung WANG, Taipei (TW); Wan-Hwa CHENG, Taipei (TW)

(21) Appl. No.: 14/814,526

(22) Filed: Jul. 31, 2015

(30) Foreign Application Priority Data
Apr. 30, 2015 (TW) 104114018
Jul. 20, 2015 (TW) 104123479
Jul. 20, 2015 (TW) 104123480

(51) Int. Cl. A61M 5/32

(52) U.S. Cl. CPC A61M 5/3216

(57) ABSTRACT

A safety syringe includes a barrel including a hollow cylindrical tip; a needle assembly including a hollow needle hub on the tip; a hinge including a bifurcated base and a moveable bifurcation on the needle hub, the movable bifurcation having two relaxation length of hinge ends pivotably secured to the base, a wall member formed with the bifurcation, and a V-shaped resilient member having two ends hingedly secured to the bifurcation and the base respectively; and a needle protection housing including a grooved body and a latch releasably secured to the wall member. When the needle protection housing is pivoted about both the resilient member and the hinge ends, resilient energy is stored in the resilient member. When the resilient energy reaches a set value, the resilient member releases the resilient energy to pivot the needle protection housing toward a hypodermic needle until the hypodermic needle is lockingly concealed in the grooved body.

### BACKGROUND OF THE INVENTION

1. Field of the Invention

The invention relates to medical syringes and more particularly to a safety syringe having a tamper-proof characteristic.

2. Description of Related Art

A major cause of the spread of infectious disease in the population is due to the presence of exposed hypodermic needles. Infection can be spread from accidental needle sticks by way of exposed, contaminated needles used in high risk patients in hospitals and medical facilities. Used syringes with extended needles present a risk to medical employees and others.

Typical safety syringes are characterized in that the exposed needle will retract at the end of an injection cycle. Further, some prior safety syringes are characterized in that the used needle is retracted in an internal concealed position and cannot be reused, i.e., being self-destructed and single use.

While a wide range of safety syringes are disclosed, the need for advancement of the art still exists.

### SUMMARY OF THE INVENTION

It is therefore one object of the invention to provide a safety syringe having a hypodermic needle which can be concealed after use. This can prevent the needle from accidentally pricking a medical employee to contaminate the medical employee with microorganisms or blood on the needle. Further, this can prevent the safety syringe from being used for a second time.

For achieving above and other objects, in a first aspect of the invention there is provided a safety syringe comprising: a barrel including a casing tube and a hollow cylindrical tip; a needle assembly including a hollow needle hub fastened on the tip, and a hypodermic needle projecting out of the needle hub and being in communication with the tip; a hinge including a bifurcated base disposed on the needle hub, a moveable bifurcation having two relaxation length of hinge ends pivotably secured to two open ends of the base respectively, a space defined between the moveable bifurcation and the base, a wall member formed with the moveable bifurcation, and a V-shaped resilient member having two ends hingedly secured to the moveable bifurcation and the base respectively; and a needle protection housing including a grooved body and a forward latch releasably secured to the wall member; wherein the safety syringe is capable of operation such that, when the needle protection housing is pivoted about both the V-shaped resilient member and the two relaxation length of hinge ends, resilient energy is stored in the V-shaped resilient member, and when the resilient energy reaches a predetermined value, the V-shaped resilient member releases the resilient energy to pivot the needle protection housing toward the hypodermic needle until the hypodermic needle is lockingly concealed in the grooved body.

Preferably, the V-shaped resilient member includes a first part having a hinge hingedly secured to the base, and a second part having a hinge hingedly secured to the moveable bifurcation, length of the first part being greater than that of the second part.

Preferably, the length of the first part is greater at least 30% than that of the second part.

Preferably, the length of the first part is two times greater than that of the second part.

Preferably, the V-shaped resilient member further includes at least one resilient enhanced rib.

Preferably, the wall member is perpendicular to the moveable bifurcation and includes a through hole, and wherein the latch is inserted through the through hole to be releasably secured thereto.

Preferably, the needle protection housing further comprises a block member at a rear end of the body, two side walls on the body, at least one locking member for needle on an inner surface of at least one of the side walls, two opposite extensions, each extending forwardly from a front end of the side wall, and a space defined by the extensions, the side walls, and the block member.

In a second aspect of the invention there is provided an arrangement for safety syringe comprising: a barrel including a casing tube, a hollow cylindrical tip, and a forward hollow cylinder containing the tip and being concentric with the tip, the hollow cylinder having internal threads; a needle assembly including a hollow needle hub fastened on the tip, a hypodermic needle projecting out of the needle hub and being in communication with the tip, and two opposite protrusions on a rear end of the needle hub, the protrusions being configured to threadedly secure to the threads; a hinge including a bifurcated base disposed on the needle hub, a ring formed with the base and being fitted onto the hollow cylinder, a moveable bifurcation having two relaxation length of hinge ends pivotably secured to two open ends of the base respectively, a space defined between the moveable bifurcation and the base, a wall member formed with the moveable bifurcation, and a V-shaped resilient member having two ends hingedly secured to the moveable bifurcation and the base respectively; and a needle protection housing including a grooved body and a forward latch releasably secured to the wall member; wherein the safety syringe is capable of operation such that, when the needle protection housing is pivoted about both the V-shaped resilient member and the two relaxation length of hinge ends, resilient energy is stored in the V-shaped resilient member, and when the resilient energy reaches a predetermined value, the V-shaped resilient member releases the resilient energy to pivot the needle protection housing toward the hypodermic needle until the hypodermic needle is lockingly concealed in the grooved body.

Preferably, the V-shaped resilient member includes a first part having a hinge hingedly secured to the base, and a second part having a hinge hingedly secured to the moveable bifurcation, length of the first part being greater than that of the second part.

Preferably, the length of the first part is greater at least 30% than that of the second part.

Preferably, the length of the first part is two times greater than that of the second part.

Preferably, the V-shaped resilient member further includes at least one resilient enhanced rib.

Preferably, the wall member is perpendicular to the moveable bifurcation and includes a through hole, and wherein the latch is inserted through the through hole to be releasably secured thereto.

Preferably, the needle protection housing further comprises a block member at a rear end of the body, two side walls on the body, at least one locking member for needle on an inner surface of at least one of the side walls, two opposite extensions, each extending forwardly from a front end of the side wall, and a space defined by the extensions, the side walls, and the block member.

Preferably, the hinge further comprises a C-ring disposed in front of the ring for clamping the hollow cylinder.

In a third aspect of the invention there is provided an arrangement for a safety syringe comprising: a barrel including a casing tube and a hollow cylindrical tip; a needle assembly including a hollow needle hub fastened on the tip, a hypodermic needle projecting out of the needle hub and being in communication with the tip, and two opposite protrusions on a rear end of the needle hub; a hinge including a bifurcated base disposed on the needle hub, a hollow cylinder formed with the base and including internal threads and a rear rim, a moveable bifurcation having two relaxation length of hinge ends pivotably secured to two open ends of the base respectively, a space defined between the moveable bifurcation and the base, a wall member formed with the moveable bifurcation, and a V-shaped resilient member having two ends hingedly secured to the moveable bifurcation and the base respectively wherein the protrusions are configured to threadedly secure to the threads and the hollow cylinder is disposed on a portion of the needle hub which is disposed on a portion of the tip; and a needle protection housing including a grooved body and a forward latch releasably secured to the wall member; wherein the safety syringe is capable of operation such that, when the needle protection housing is pivoted about both the V-shaped resilient member and the two relaxation length of hinge ends, resilient energy is stored in the V-shaped resilient member, and when the resilient energy reaches a predetermined value, the V-shaped resilient member releases the resilient energy to pivot the needle protection housing toward the hypodermic needle until the hypodermic needle is lockingly concealed in the grooved body.

Preferably, the V-shaped resilient member includes a first part having a hinge hingedly secured to the base, and a second part having a hinge hingedly secured to the moveable bifurcation, length of the first part being greater than that of the second part.

Preferably, the length of the first part is greater at least 30% than that of the second part.

Preferably, the length of the first part is two times greater than that of the second part.

Preferably, the V-shaped resilient member further includes at least one resilient enhanced rib.

Preferably, the wall member is perpendicular to the moveable bifurcation and includes a through hole, and wherein the latch is inserted through the through hole to be releasably secured thereto.

Preferably, the needle protection housing further comprises a block member at a rear end of the body, two side walls on the body, at least one locking member for needle on an inner surface of at least one of the side walls, two opposite extensions, each extending forwardly from a front end of the side wall, and a space defined by the extensions, the side walls, and the block member.

Preferably, the hinge further includes a clip ring fastened between the tip and the hollow cylinder and urging against the rim.

In a fourth aspect of the invention there is provided an arrangement for a safety syringe comprising: a barrel including a casing tube and a hollow cylindrical tip; a needle assembly including a hollow needle hub fastened on the tip, and a hypodermic needle projecting out of the needle hub and being in communication with the tip; a hinge including a bifurcated base, a hollow cylinder formed with the base and having a hollow cylindrical member projecting out of a front end, and two opposite protuberances formed at a rear end, a moveable bifurcation having two relaxation length of hinge ends pivotably secured to two open ends of the base respectively, a space defined between the moveable bifurcation and the base, a wall member formed with the moveable bifurcation, and a V-shaped resilient member having two ends hingedly secured to the moveable bifurcation and the base respectively wherein the cylindrical member is fastened in the needle hub; and a needle protection housing including a grooved body and a forward latch releasably secured to the wall member; wherein the safety syringe is capable of operation such that, when the needle protection housing is pivoted about both the V-shaped resilient member and the two relaxation length of hinge ends, resilient energy is stored in the V-shaped resilient member, and when the resilient energy reaches a predetermined value, the V-shaped resilient member releases the resilient energy to pivot the needle protection housing toward the hypodermic needle until the hypodermic needle is lockingly concealed in the grooved body.

Preferably, the V-shaped resilient member includes a first part having a hinge hingedly secured to the base, and a second part having a hinge hingedly secured to the moveable bifurcation, length of the first part being greater than that of the second part.

Preferably, the length of the first part is greater at least 30% than that of the second part.

Preferably, the length of the first part is two times greater than that of the second part.

Preferably, the V-shaped resilient member further includes at least one resilient enhanced rib.

Preferably, the wall member is perpendicular to the moveable bifurcation and includes a through hole, and wherein the latch is inserted through the through hole to be releasably secured thereto.

Preferably, the needle protection housing further comprises a block member at a rear end of the body, two side walls on the body, at least one locking member for needle on an inner surface of at least one of the side walls, two opposite extensions, each extending forwardly from a front end of the side wall, and a space defined by the extensions, the side walls, and the block member.

The above and other objects, features and advantages of the invention will become apparent from the following detailed description taken with the accompanying drawings.

### BRIEF DESCRIPTION OF THE DRAWINGS

FIG. 1 is a perspective view of a safety syringe according to a first preferred embodiment of the invention;

FIG. 2 is an exploded view of the safety syringe;

FIG. 3 is a view similar to FIG. 1 showing the needle protection housing pivoted to cover the needle assembly after use and prior to disposal;

FIG. 4A is a side elevation of FIG. 1 in part section;

FIG. 4B is a view similar to FIG. 4A showing the needle protection housing being pivoted counterclockwise in a first stage of needle covering operation after use;

FIG. 4C is a view similar to FIG. 4B showing the needle protection housing being pivoted counterclockwise in a second stage of needle covering operation after use;

FIG. 4D is a view similar to FIG. 4C showing the needle protection housing being pivoted counterclockwise in a final stage of needle covering operation after use;

FIG. 5 is a perspective view of a safety syringe according to a second preferred embodiment of the invention;

FIG. 6 is an exploded view of the safety syringe of FIG. 5;

FIG. 7 is a view similar to FIG. 5 showing the needle protection housing pivoted to cover the needle assembly after use and prior to disposal;

FIG. 8 is a side elevation of FIG. 5;

FIG. 9 is a view similar to FIG. 8 showing the needle protection housing being pivoted counterclockwise to cover the needle after use;

FIG. 10 is a perspective view of a safety syringe according to a third preferred embodiment of the invention;

FIG. 11A is an exploded view of the safety syringe of FIG. 10;

FIG. 11B is a further exploded view of the safety syringe of FIG. 10;

FIG. 12 is a view similar to FIG. 10 showing the needle protection housing pivoted to cover the needle assembly after use and prior to disposal;

FIG. 13 is a side elevation of FIG. 10 in part section;

FIG. 14 is a view similar to FIG. 13 showing the needle protection housing being pivoted counterclockwise to cover the needle after use;

FIG. 15 is a perspective view of a safety syringe according to a fourth preferred embodiment of the invention;

FIG. 16 is an exploded view of the safety syringe of FIG. 15; and

FIG. 17 is a side elevation of FIG. 15 in part section.

### DETAILED DESCRIPTION OF THE INVENTION

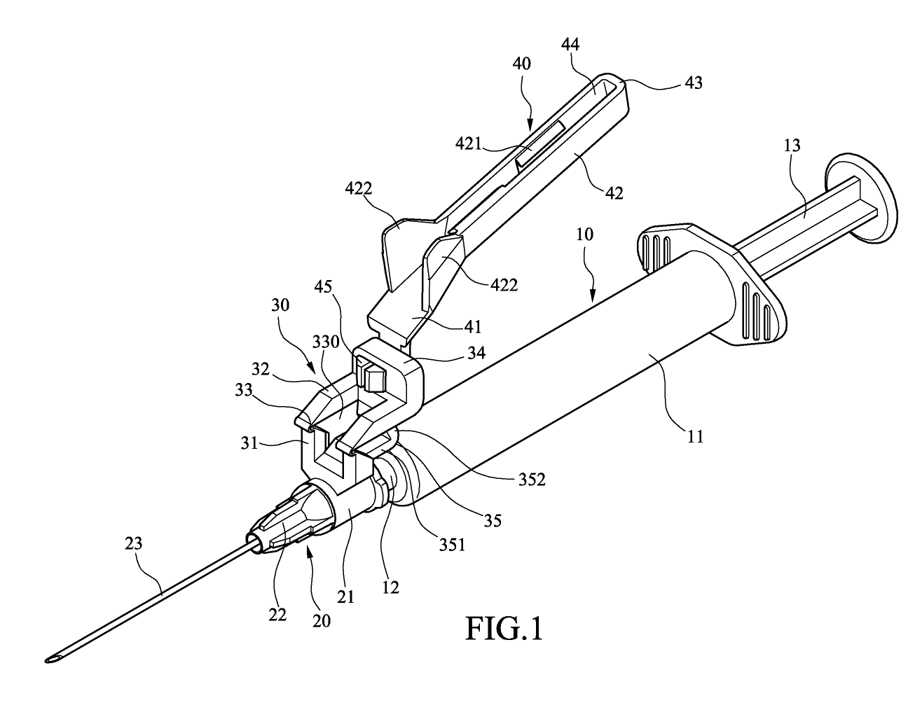

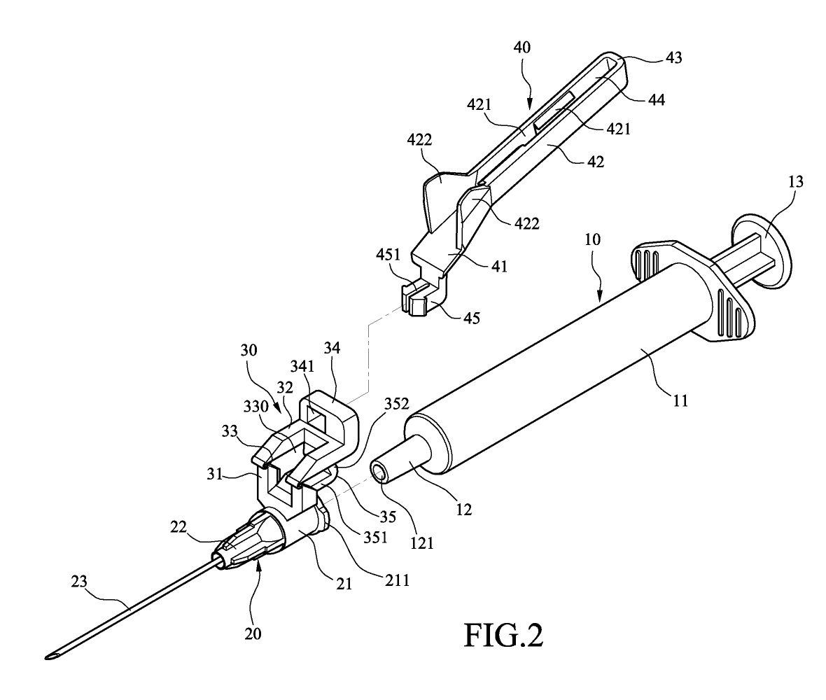

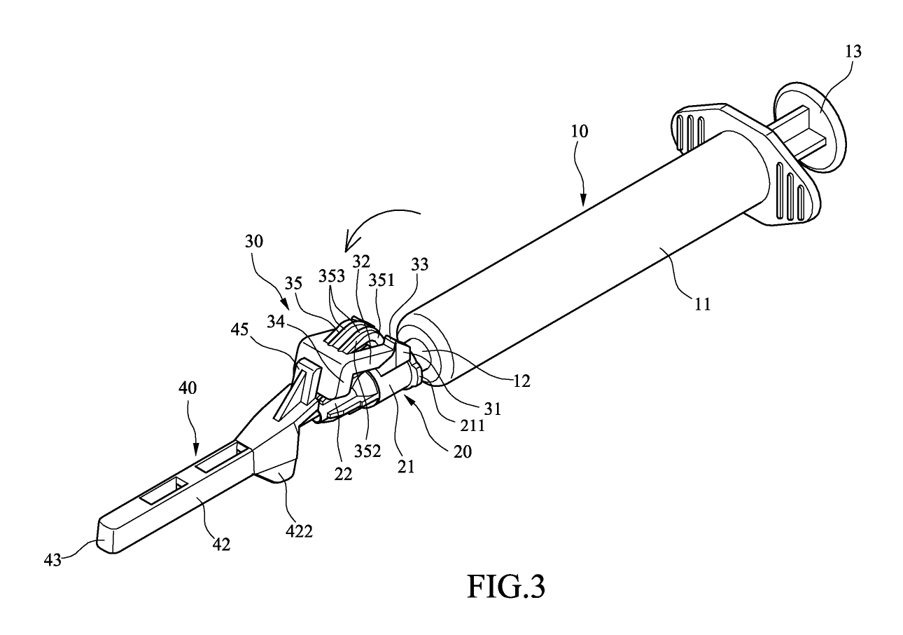

Referring to FIGS. 1 to 4D, a safety syringe in accordance with a first preferred embodiment of the invention comprises a barrel 10, a needle assembly 20, a hinge 30, and a needle protection housing 40 as discussed in detail below.

The barrel 10 includes a cylindrical casing tube 11, a cylindrical tip 12 having a diameter less than that of the casing tube 11 and a forward opening 121, and a stem 13. The needle assembly 20 includes a hollow needle hub 21 having a rear flange 211, the needle hub 21 fastened on the tip 12, a support 22 formed with a front end of the needle hub 21, and a hypodermic needle 23 projecting out of the support 22 and being in communication with the tip 12 via the needle hub 21.

The hinge 30 includes a bifurcated base 31 disposed on the circumferential surface of the needle hub 21, a moveable bifurcation 32 having two relaxation length of hinge ends 33 pivotably secured to two open ends of the base 31, a space 330 defined between the moveable bifurcation 32 and the base 31, a wall member 34 formed with the moveable bifurcation 32, the wall member 34 being perpendicular to the moveable bifurcation 32 and including a through hole 341, and a V-shaped resilient member 35 including a first part 351 having a hinge 3511 hingedly secured to the body of the base 31, a second part 352 having a hinge 3521 hingedly secured to the body of the moveable bifurcation 32, and a plurality of resilient enhanced ribs 353. The first part 351 is longer than the second part 352. Preferably, the first part 351 is longer at least 30% than the second part 352. More preferably, the first part 351 is twice as long as the second part 352. This, as the subject of the invention, can greatly increase the clamping force of the hinge 30 exerted on the needle protection housing 40 as discussed later.

The needle protection housing 40 includes a grooved body 41 having a rear end 43, two side walls 42 on the body 41, a locking member 421 on an inner surface of the side wall 42, two opposite extensions 422 each extending forwardly from a front end of the side wall 42, a space 44 defined by the extensions 422, the side walls 42, and the rear end 43, and a latch 45 at a forward end of the body 41, a slit 451 formed through the latch 45. The latch 45 is inserted through the through hole 341 to fasten the needle protection housing 40 and the hinge 30 together.

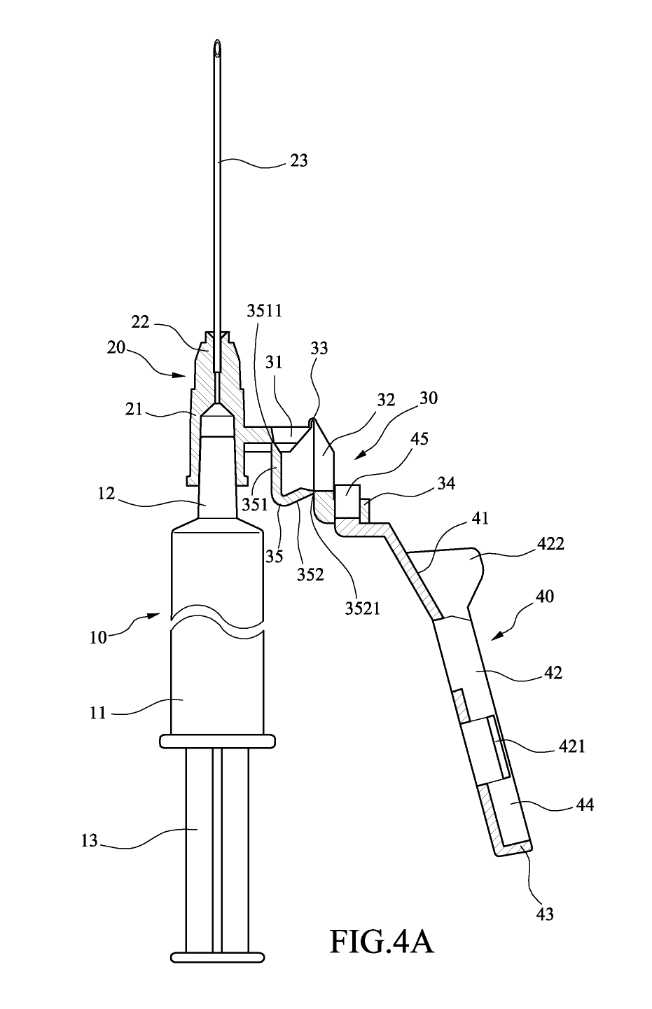

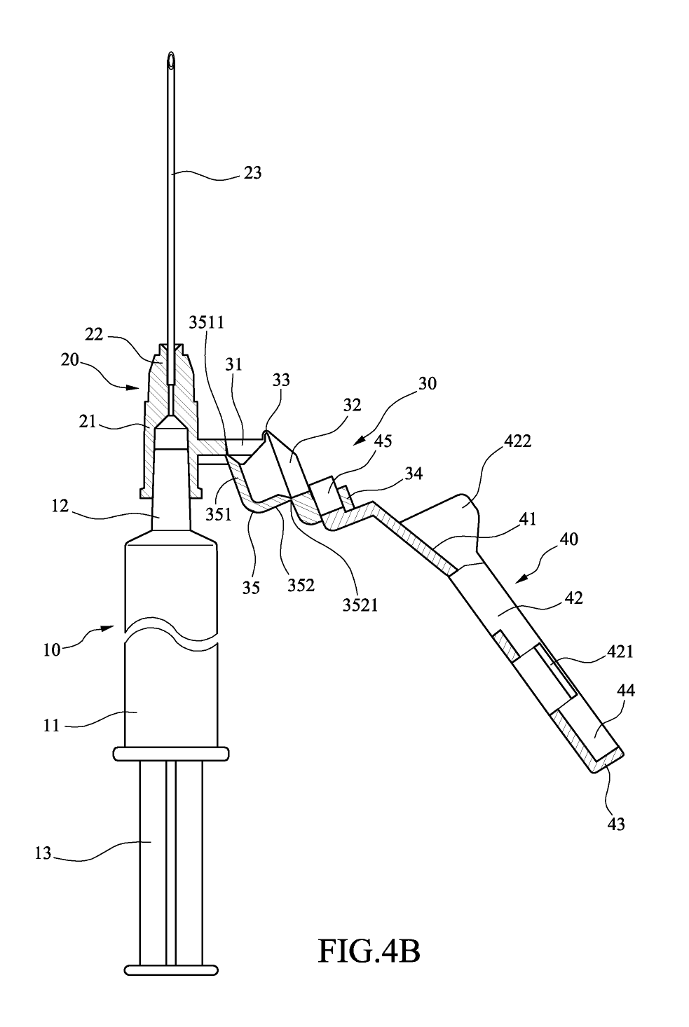

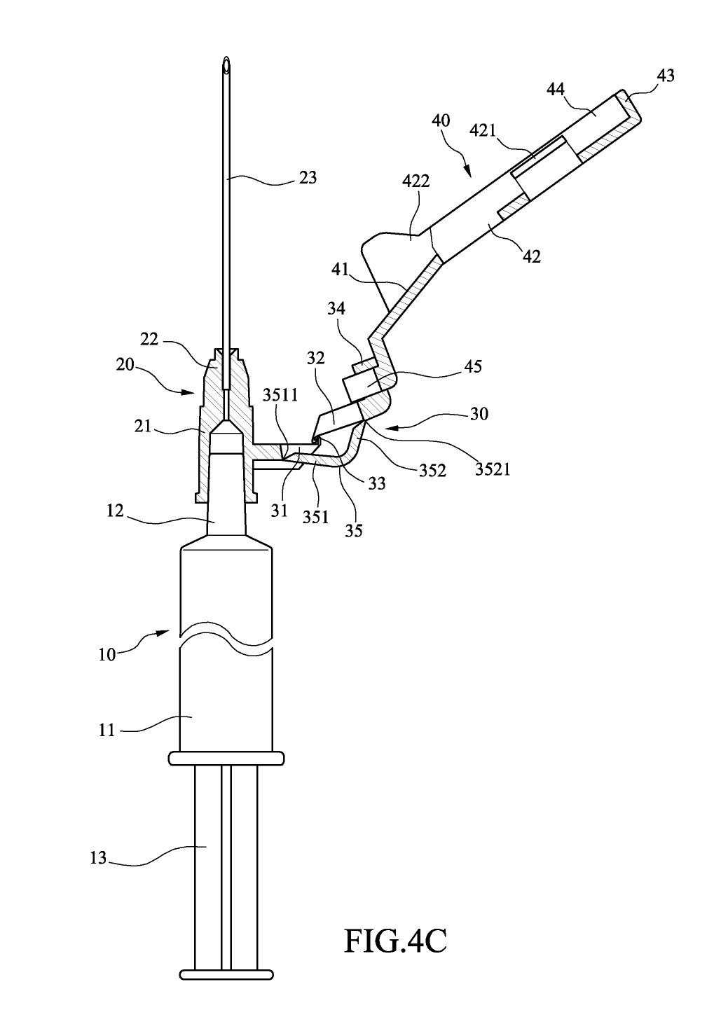

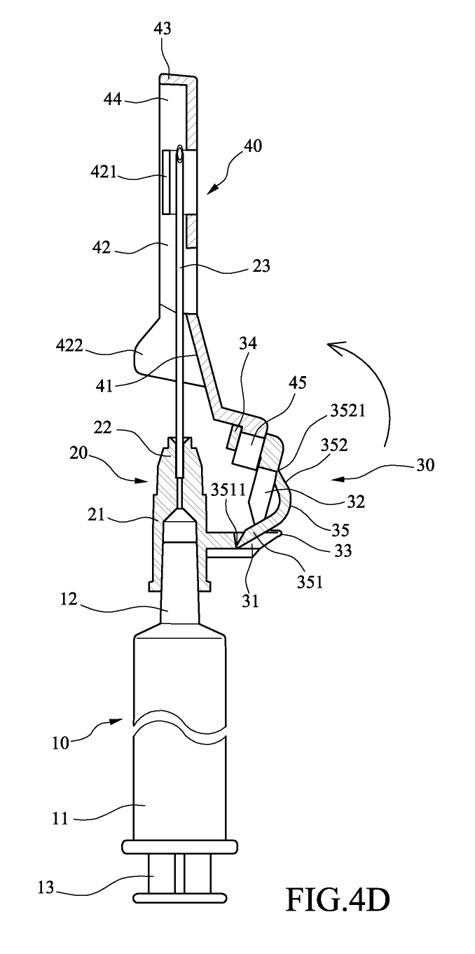

In a ready to administer injections state, an angle of the second part 352 with respect to the first part 351 is about 35-degree (see FIG. 4A). After use, a medical employee may use one hand to hold the barrel 10 and the other hand to hold and pivot the needle protection housing 40 about both the hinges 3511 and the two relaxation length of hinge ends 33 with the angle increased to about 87-degree (see FIG. 4B). In the meantime, resilient energy is stored in the V-shaped resilient member 35. And in turn, the energized V-shaped resilient member 35 releases its resilient energy immediately after the angle reaches about 100-degree (see FIG. 4C). From this point of no return, the needle protection housing 40 is activated by the released energy of the V-shaped resilient member 35 to automatically pivot toward the hypodermic needle 23 about both the two relaxation length of hinge ends 33 and the hinges 3511 until the locking member 421 passes the hypodermic needle 23 to be concealed in the space 44. In this position, the hypodermic needle 23 is locked. Thus, it can prevent the hypodermic needle 23 from accidentally pricking the medical employee to contaminate the medical employee with microorganisms or blood on the hypodermic needle 23. Further, this can prevent the safety syringe from being used for a second time. Therefore, the safety syringe is ready for disposal.

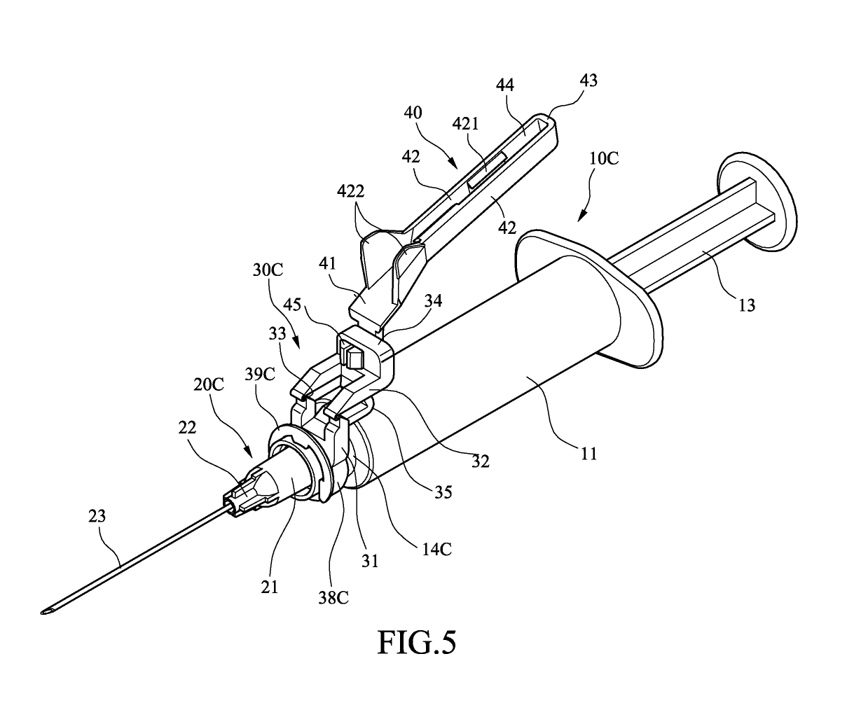

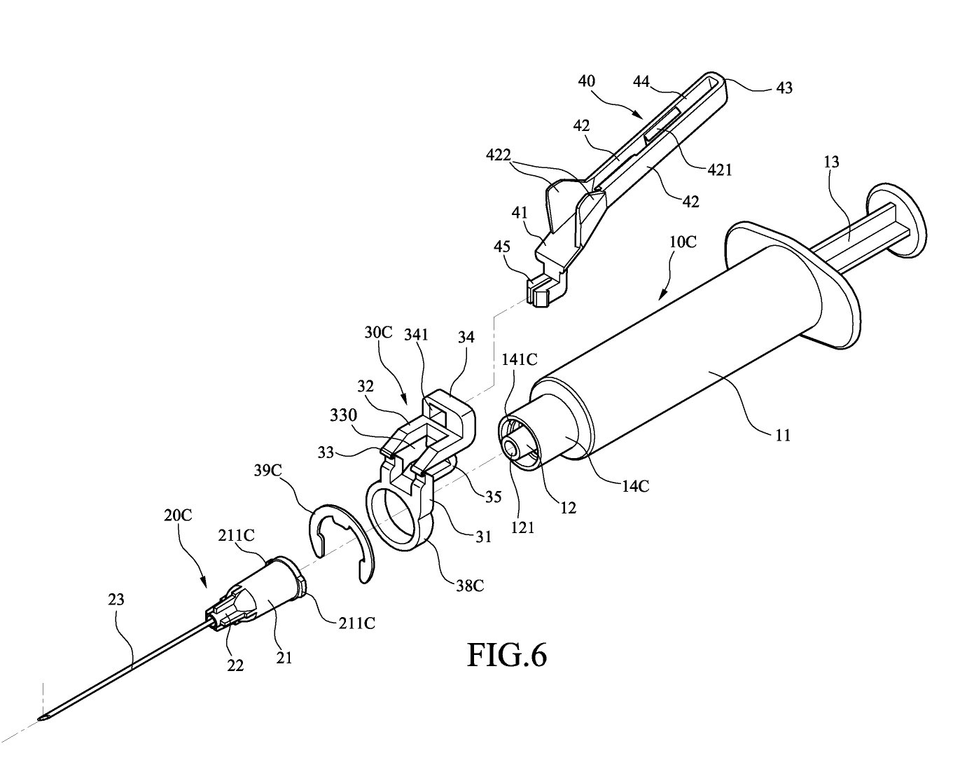

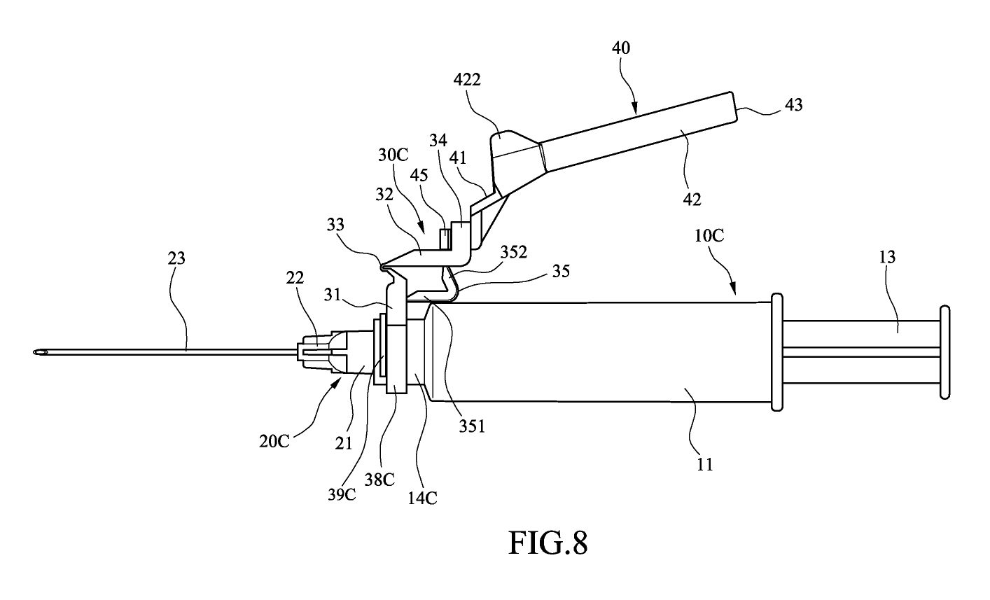

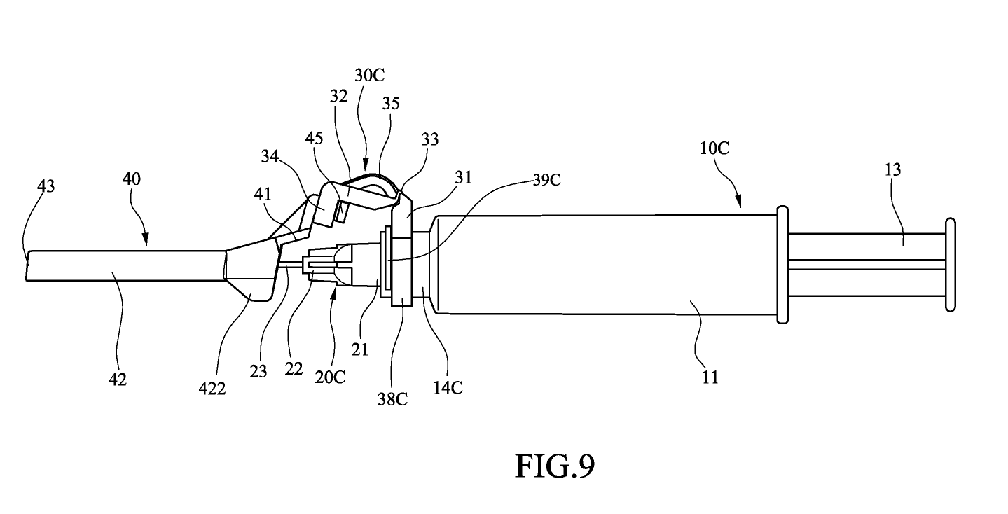

Referring to FIGS. 5 to 9, a safety syringe in accordance with a second preferred embodiment of the invention is shown. The characteristics of the second preferred embodiment are substantially the same as that of the first preferred embodiment except the following: The barrel 10C includes a forward hollow cylinder 14C containing the tip 12 and being concentric with the tip 12, the hollow cylinder 14C having internal threads 141C. The needle hub 21 of the needle assembly 20C includes two opposite protrusions 211C on a rear end adapted to threadedly secure to the threads 141C. The hinge 30C includes a ring 38C formed with the base 31, the ring 38C being fitted onto the hollow cylinder 14C, and a C-ring 39C disposed in front of the ring 38C and clamping the hollow cylinder 14C for fastening purpose.

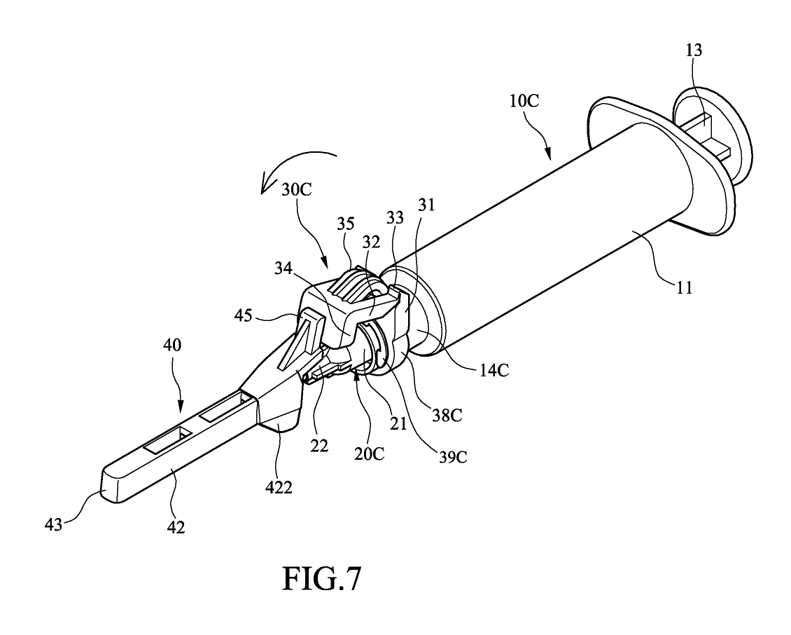

In a ready to administer injections state by referring to FIGS. 7 to 9, an angle of the second part 352 with respect to the first part 351 is about 35-degree. After use, a medical employee may use one hand to hold the barrel 10C and the other hand to hold and pivot the needle protection housing 40 about both the hinges 3511 and the two relaxation length of hinge ends 33 with the angle increased to about 87-degree. In the meantime, resilient energy is stored in the V-shaped resilient member 35. And in turn, the energized V-shaped resilient member 35 releases its resilient energy immediately after the angle reaches about 100-degree. From this point of no return, the needle protection housing 40 is activated by the released energy of the V-shaped resilient member 35 to automatically pivot toward the hypodermic needle 23 about both the two relaxation length of hinge ends 33 and the hinges 3511 until the locking member 421 passes the hypodermic needle 23 to be concealed in the space 44. In this position, the hypodermic needle 23 is locked. Thus, it can prevent the hypodermic needle 23 from accidentally pricking the medical employee to contaminate the medical employee with microorganisms or blood on the hypodermic needle 23. Further, this can prevent the safety syringe from being used for a second time. Therefore, the safety syringe is ready for disposal.

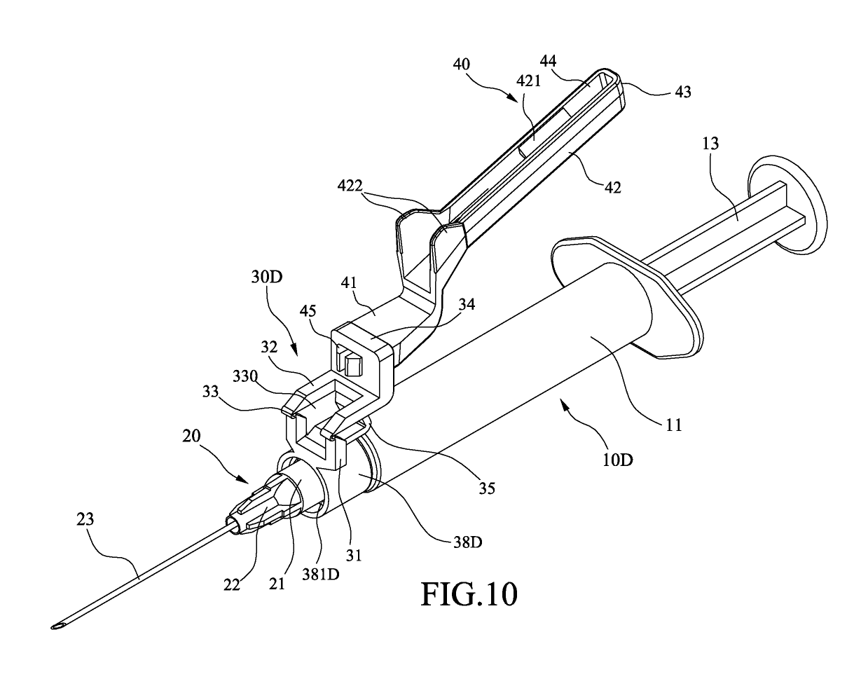

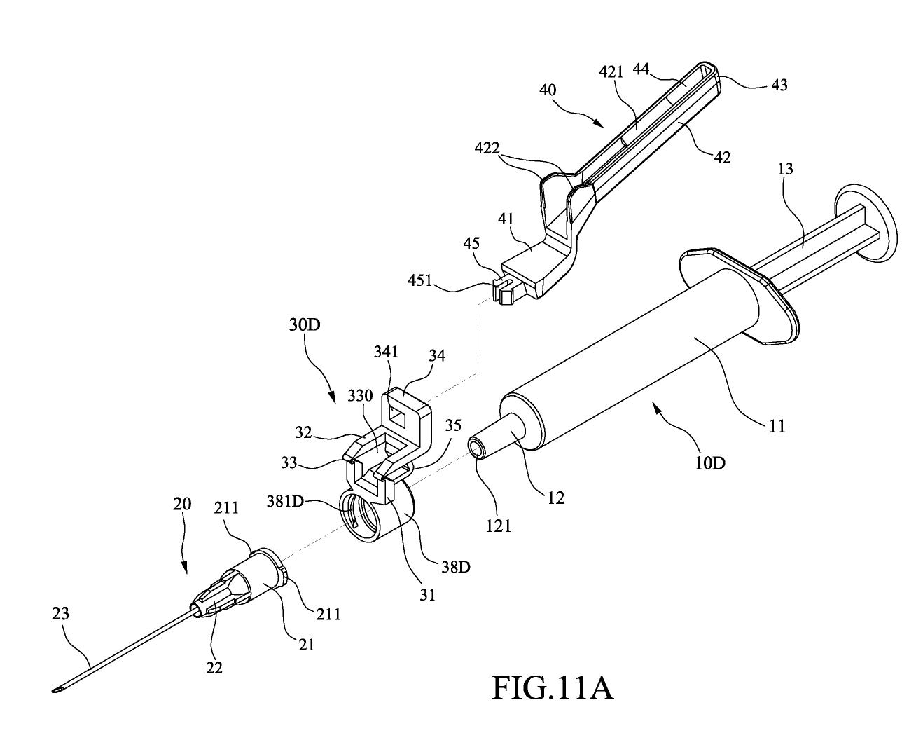

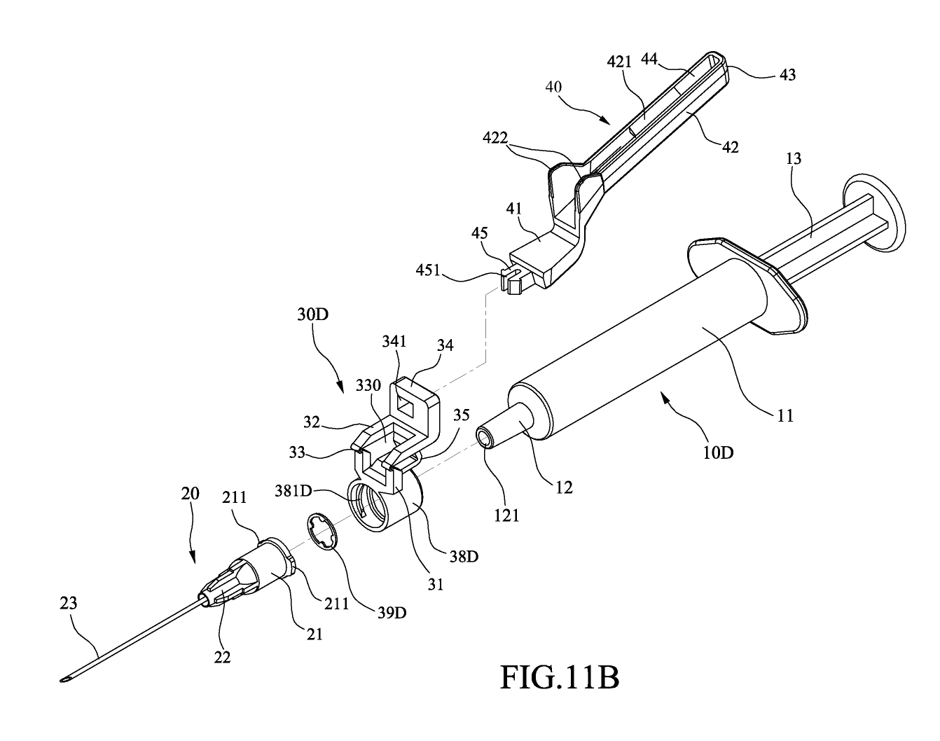

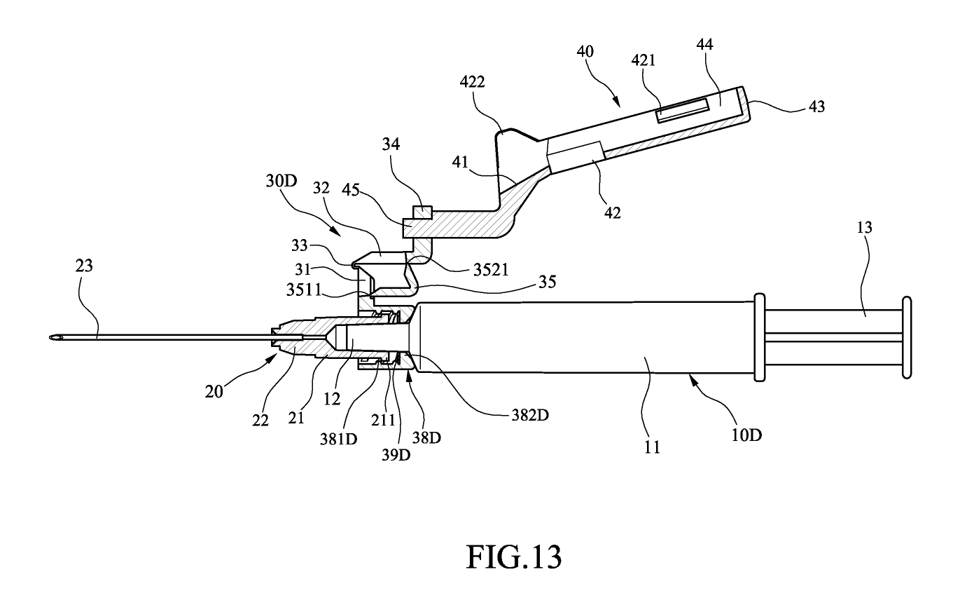

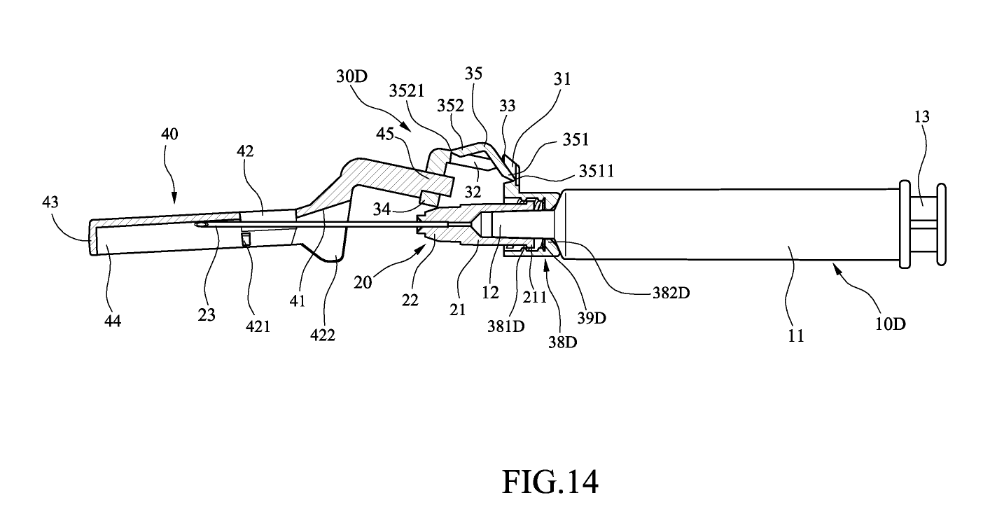

Referring to FIGS. 10 to 14, a safety syringe in accordance with a third preferred embodiment of the invention is shown. The characteristics of the third preferred embodiment are substantially the same as that of the first preferred embodiment except the following: The hinge 30D includes a hollow cylinder 38D formed with the base 31 and having internal threads 381D, a clip ring 39D, and a rim 382D formed on a rear end of the hollow cylinder 38D. The needle hub 21 of the needle assembly 20D includes two opposite protrusions 422 on a rear end adapted to threadedly secure to the threads 381D. The hollow cylinder 38D is disposed around the tip 12 with the clip ring 39D fastened between the tip 12 and the hollow cylinder 38D. The tip 12 is fastened in the needle hub 21.

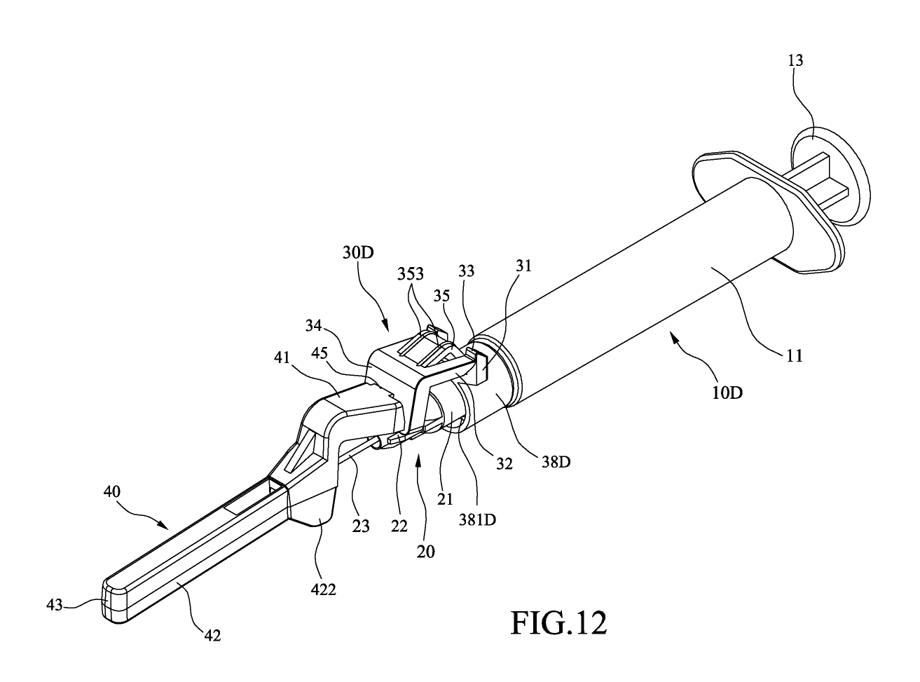

In a ready to administer injections state by referring to FIGS. 12 to 14, an angle of the second part 352 with respect to the first part 351 is about 35-degree. After use, a medical employee may use one hand to hold the barrel 10D and the other hand to hold and pivot the needle protection housing 40 about both the hinges 3511 and the two relaxation length of hinge ends 33 with the angle increased to about 87-degree. In the meantime, resilient energy is stored in the V-shaped resilient member 35. And in turn, the energized V-shaped resilient member 35 releases its resilient energy immediately after the angle reaches about 100-degree. From this point of no return, the needle protection housing 40 is activated by the released energy of the V-shaped resilient member 35 to automatically pivot toward the hypodermic needle 23 about both the two relaxation length of hinge ends 33 and the hinges 3511 until the locking member 421 passes the hypodermic needle 23 to be concealed in the space 44. In this position, the hypodermic needle 23 is locked. Thus, it can prevent the hypodermic needle 23 from accidentally pricking the medical employee to contaminate the medical employee with microorganisms or blood on the hypodermic needle 23. Further, this can prevent the safety syringe from being used for a second time. Therefore, the safety syringe is ready for disposal.

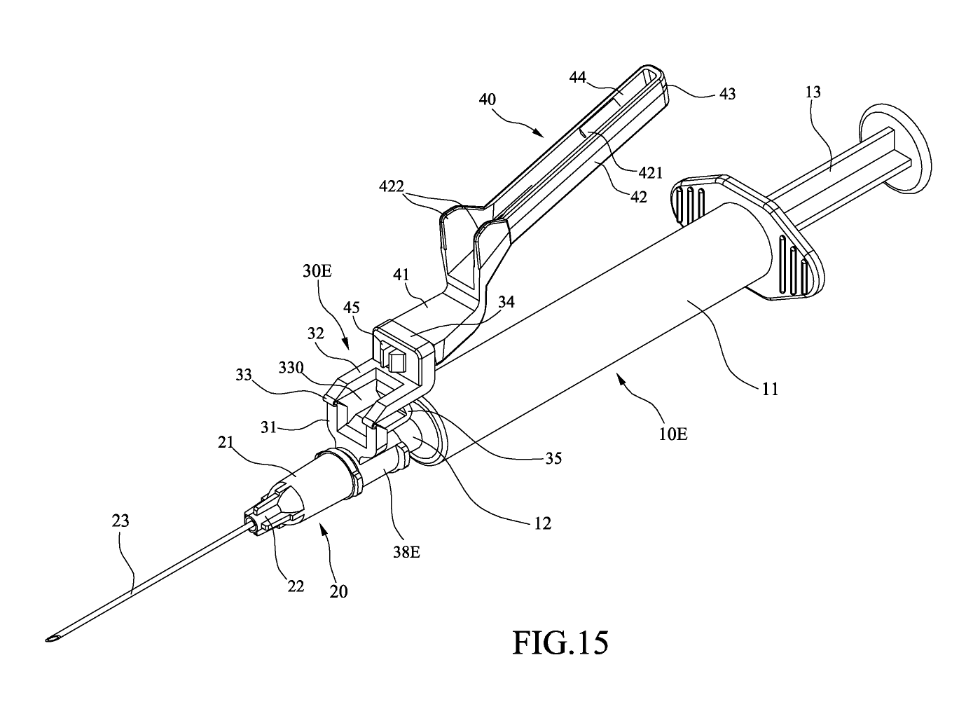

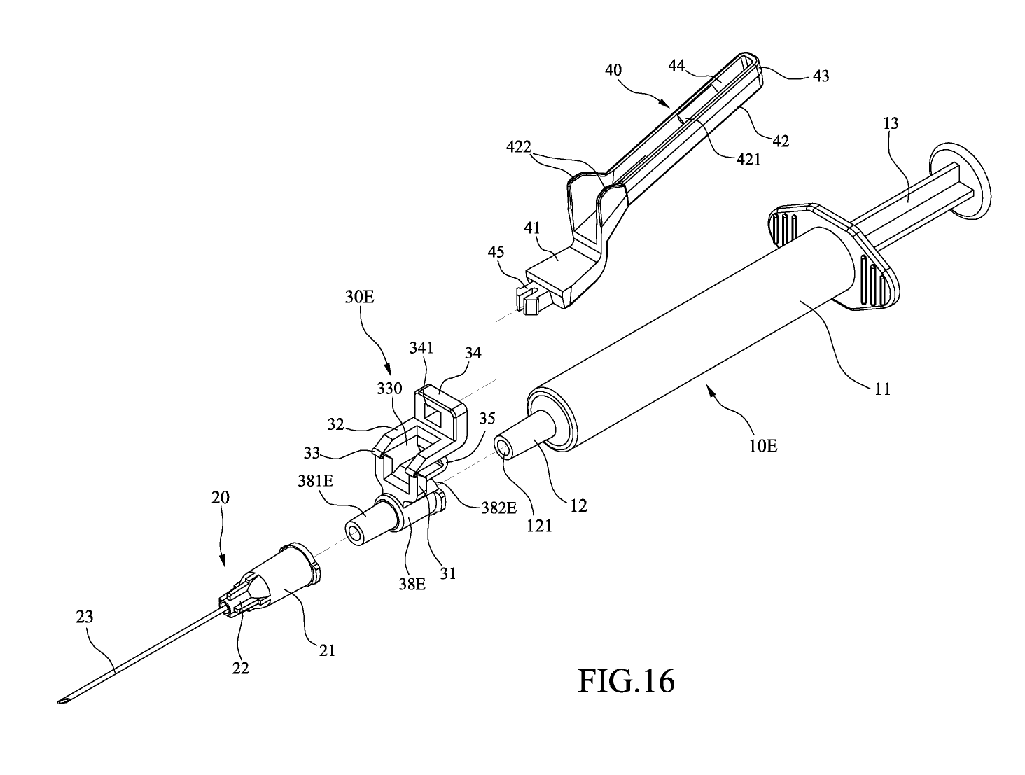

Referring to FIGS. 15 to 17, a safety syringe in accordance with a fourth preferred embodiment of the invention is shown. The characteristics of the fourth preferred embodiment are substantially the same as that of the first preferred embodiment except the following: The hinge 30E includes a hollow cylinder 38E formed with the base 31. A hollow cylindrical member 381E is formed at a front end of the hollow cylinder 38E. Two opposite protuberances 382E are formed at a rear end of the hollow cylinder 38E. The cylindrical member 381E is inserted into the needle hub 21 for fastening with the rear end of the needle hub 21 urging against a shoulder between the cylindrical member 381E and the hollow cylinder 38E.

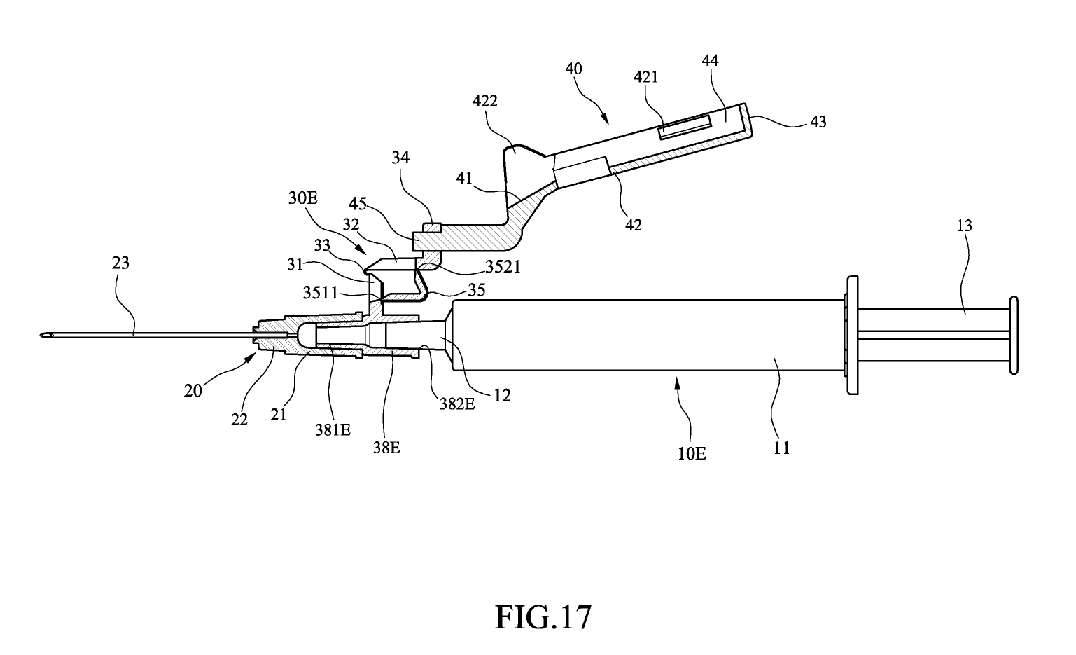

In a ready to administer injections state by referring to FIGS. 15 and 17, an angle of the second part 352 with respect to the first part 351 is about 35-degree. After use, a medical employee may use one hand to hold the barrel 10E and the other hand to hold and pivot the needle protection housing 40 about both the hinges 3511 and the two relaxation length of hinge ends 33 with the angle increased to about 87-degree. In the meantime, resilient energy is stored in the V-shaped resilient member 35. And in turn, the energized V-shaped resilient member 35 releases its resilient energy immediately after the angle reaches about 100-degree. From this point of no return, the needle protection housing 40 is activated by the released energy of the V-shaped resilient member 35 to automatically pivot toward the hypodermic needle 23 about both the two relaxation length of hinge ends 33 and the hinges 3511 until the locking member 421 passes the hypodermic needle 23 to be concealed in the space 44. In this position, the hypodermic needle 23 is locked. Thus, it can prevent the hypodermic needle 23 from accidentally pricking the medical employee to contaminate the medical employee with microorganisms or blood on the hypodermic needle 23. Further, this can prevent the safety syringe from being used for a second time. Therefore, the safety syringe is ready for disposal.

While the invention has been described in terms of preferred embodiments, those skilled in the art will recognize that the invention can be practiced with modifications within the spirit and scope of the appended claims.

What is claimed is:

1. A safety syringe comprising:
a barrel (10) including a casing tube (11) and a hollow cylindrical tip (12);
a needle assembly (20) including a hollow needle hub (21) fastened on the tip (12), and a hypodermic needle (23) projecting out of the needle hub (21) and being in communication with the tip (12);
a hinge (30) including a bifurcated base (31) disposed on the needle hub (21), a moveable bifurcation (32) having two relaxation length of hinge ends (33) pivotably secured to two open ends of the base (31) respectively, a space (330) defined between the moveable bifurcation (32) and the base (31), a wall member (34) formed with the moveable bifurcation (32), and a V-shaped resilient member (35) having two ends hingedly secured to the moveable bifurcation (32) and the base (31) respectively; and
a needle protection housing (40) including a grooved body (41) and a forward latch (45) releasably secured to the wall member (34);
wherein the safety syringe is capable of operation such that, when the needle protection housing (40) is pivoted about both the V-shaped resilient member (35) and the two relaxation length of hinge ends (33), resilient energy is stored in the V-shaped resilient member (35), and when the resilient energy reaches a predetermined value, the V-shaped resilient member (35) releases the resilient energy to pivot the needle protection housing (40) toward the hypodermic needle (23) until the hypodermic needle (23) is lockingly concealed in the grooved body (41).

2. The safety syringe of claim 1, wherein the V-shaped resilient member (35) includes a first part (351) having a hinge (3511) hingedly secured to the base (31), and a second part (352) having a hinge (3521) hingedly secured to the moveable bifurcation (32), length of the first part (351) being greater than that of the second part (352).

3. The safety syringe of claim 2, wherein the length of the first part (351) is greater at least 30% than that of the second part (352).

4. The safety syringe of claim 2, wherein the length of the first part (351) is two times greater than that of the second part (352).

5. The safety syringe of claim 2, wherein the V-shaped resilient member (35) further includes at least one resilient enhanced rib (353).

6. The safety syringe of claim 3, wherein the V-shaped resilient member (35) further includes at least one resilient enhanced rib (353).

7. The safety syringe of claim 1, wherein the wall member (34) is perpendicular to the moveable bifurcation (32) and includes a through hole (341), and wherein the latch (45) is inserted through the through hole (341) to be releasably secured thereto.

8. The safety syringe of claim 1, wherein the needle protection housing (40) further comprises a block member (43) at a rear end of the body (41), two side walls (42) on the body (41), at least one locking member (421) for needle (23) on an inner surface of at least one of the side walls (42), two opposite extensions (422), each extending forwardly from a front end of the side wall (42), and a space (44) defined by the extensions (422), the side walls (42), and the block member (43).

9. A safety syringe comprising:
a barrel (10C) including a casing tube (11), a hollow cylindrical tip (12), and a forward hollow cylinder (14C) containing the tip (12) and being concentric with the tip (12), the hollow cylinder (14C) having internal threads (141C);
a needle assembly (20C) including a hollow needle hub (21) fastened on the tip (12), a hypodermic needle (23) projecting out of the needle hub (21) and being in communication with the tip (12), and two opposite protrusions (211C) on a rear end of the needle hub (21), the protrusions (211C) being configured to threadedly secure to the threads (141C);
a hinge (30C) including a bifurcated base (31) disposed on the needle hub (21), a ring (38C) formed with the base (31) and being fitted onto the hollow cylinder (14C), a moveable bifurcation (32) having two relaxation length of hinge ends (33) pivotably secured to two open ends of the base (31) respectively, a space (330) defined between the moveable bifurcation (32) and the base (31), a wall member (34) formed with the moveable bifurcation (32), and a V-shaped resilient member (35) having two ends hingedly secured to the moveable bifurcation (32) and the base (31) respectively; and
a needle protection housing (40) including a grooved body (41) and a forward latch (45) releasably secured to the wall member (34);
wherein the safety syringe is capable of operation such that, when the needle protection housing (40) is pivoted about both the V-shaped resilient member (35) and the two relaxation length of hinge ends (33), resilient energy is stored in the V-shaped resilient member (35), and when the resilient energy reaches a predetermined value, the V-shaped resilient member (35) releases the resilient energy to pivot the needle protection housing (40) toward the hypodermic needle (23) until the hypodermic needle (23) is lockingly concealed in the grooved body (41).

10. The safety syringe of claim 9, wherein the V-shaped resilient member (35) includes a first part (351) having a hinge (3511) hingedly secured to the base (31), and a second part (352) having a hinge (3521) hingedly secured to the moveable bifurcation (32), length of the first part (351) being greater than that of the second part (352).

11. The safety syringe of claim 10, wherein the length of the first part (351) is greater at least 30% than that of the second part (352).

12. The safety syringe of claim 10, wherein the length of the first part (351) is two times greater than that of the second part (352).

13. The safety syringe of claim 11, wherein the V-shaped resilient member (35) further includes at least one resilient enhanced rib (353).

14. The safety syringe of claim 12, wherein the V-shaped resilient member (35) further includes at least one resilient enhanced rib (353).

15. The safety syringe of claim 9, wherein the wall member (34) is perpendicular to the moveable bifurcation (32) and includes a through hole (341), and wherein the latch (45) is inserted through the through hole (341) to be releasably secured thereto.

16. The safety syringe of claim 9, wherein the needle protection housing (40) further comprises a block member (43) at a rear end of the body (41), two side walls (42) on the body (41), at least one locking member (421) for needle (23) on an inner surface of at least one of the side walls (42), two opposite extensions (422), each extending forwardly from a front end of the side wall (42), and a space (44) defined by the extensions (422), the side walls (42), and the block member (43).

17. The safety syringe of claim 9, wherein the hinge (30C) further comprises a C-ring (39C) disposed in front of the ring (38C) for clamping the hollow cylinder (14C).

18. A safety syringe comprising:
a barrel (10D) including a casing tube (11) and a hollow cylindrical tip (12);
a needle assembly (20) including a hollow needle hub (21) fastened on the tip (12), a hypodermic needle (23) projecting out of the needle hub (21) and being in communication with the tip (12), and two opposite protrusions (211) on a rear end of the needle hub (21);
a hinge (30D) including a bifurcated base (31) disposed on the needle hub (21), a hollow cylinder (38D) formed with the base (31) and including internal threads (381D) and a rear rim (382D), a moveable bifurcation (32) having two relaxation length of hinge ends (33) pivotably secured to two open ends of the base (31) respectively, a space (330) defined between the moveable bifurcation (32) and the base (31), a wall member (34) formed with the moveable bifurcation (32), and a V-shaped resilient member (35) having two ends hingedly secured to the moveable bifurcation (32) and the base (31) respectively wherein the protrusions (211) are configured to threadedly secure to the threads (381D) and the hollow cylinder (38D) is disposed on a portion of the needle hub (21) which is disposed on a portion of the tip (12); and
a needle protection housing (40) including a grooved body (41) and a forward latch (45) releasably secured to the wall member (34);
wherein the safety syringe is capable of operation such that, when the needle protection housing (40) is pivoted about both the V-shaped resilient member (35) and the two relaxation length of hinge ends (33), resilient energy is stored in the V-shaped resilient member (35), and when the resilient energy reaches a predetermined value, the V-shaped resilient member (35) releases the resilient energy to pivot the needle protection housing (40) toward the hypodermic needle (23) until the hypodermic needle (23) is lockingly concealed in the grooved body (41).

19. The safety syringe of claim 18, wherein the V-shaped resilient member (35) includes a first part (351) having a hinge (3511) hingedly secured to the base (31), and a second part (352) having a hinge (3521) hingedly secured to the moveable bifurcation (32), length of the first part (351) being greater than that of the second part (352).

20. The safety syringe of claim 19, wherein the length of the first part (351) is greater at least 30% than that of the second part (352).

21. The safety syringe of claim 19, wherein the length of the first part (351) is two times greater than that of the second part (352).

22. The safety syringe of claim 19, wherein the V-shaped resilient member (35) further includes at least one resilient enhanced rib (353).

23. The safety syringe of claim 21, wherein the V-shaped resilient member (35) further includes at least one resilient enhanced rib (353).

24. The safety syringe of claim 18, wherein the wall member (34) is perpendicular to the moveable bifurcation (32) and includes a through hole (341), and wherein the latch (45) is inserted through the through hole (341) to be releasably secured thereto.

25. The safety syringe of claim 18, wherein the needle protection housing (40) further comprises a block member (43) at a rear end of the body (41), two side walls (42) on the body (41), at least one locking member (421) for needle (23) on an inner surface of at least one of the side walls (42), two opposite extensions (422), each extending forwardly from a front end of the side wall (42), and a space (44) defined by the extensions (422), the side walls (42), and the block member (43).

26. The safety syringe of claim 18, wherein the hinge (30D) further includes a clip ring (39D) fastened between the tip (12) and the hollow cylinder (38D) and urging against the rim (382D).

27. A safety syringe comprising:
a barrel (10E) including a casing tube (11) and a hollow cylindrical tip (12);
a needle assembly (20) including a hollow needle hub (21) fastened on the tip (12), and a hypodermic needle (23) projecting out of the needle hub (21) and being in communication with the tip (12);
a hinge (30E) including a bifurcated base (31), a hollow cylinder (38E) formed with the base (31) and having a hollow cylindrical member (381E) projecting out of a front end, and two opposite protuberances (382E) formed at a rear end, a moveable bifurcation (32) having two relaxation length of hinge ends (33) pivotably secured to two open ends of the base (31) respectively, a space (330) defined between the moveable bifurcation (32) and the base (31), a wall member (34) formed with the moveable bifurcation (32), and a V-shaped resilient member (35) having two ends hingedly secured to the moveable bifurcation (32) and the base (31) respectively wherein the cylindrical member (381E) is fastened in the needle hub (21); and
a needle protection housing (40) including a grooved body (41) and a forward latch (45) releasably secured to the wall member (34);
wherein the safety syringe is capable of operation such that, when the needle protection housing (40) is pivoted about both the V-shaped resilient member (35) and the two relaxation length of hinge ends (33), resilient energy is stored in the V-shaped resilient member (35), and when the resilient energy reaches a predetermined value, the V-shaped resilient member (35) releases the resilient energy to pivot the needle protection housing (40) toward the hypodermic needle (23) until the hypodermic needle (23) is lockingly concealed in the grooved body (41).

28. The safety syringe of claim 27, wherein the V-shaped resilient member (35) includes a first part (351) having a hinge (3511) hingedly secured to the base (31), and a second part (352) having a hinge (3521) hingedly secured to the moveable bifurcation (32), length of the first part (351) being greater than that of the second part (352).

29. The safety syringe of claim 28, wherein the length of the first part (351) is greater at least 30% than that of the second part (352).

30. The safety syringe of claim 28, wherein the length of the first part (351) is two times greater than that of the second part (352).

31. The safety syringe of claim 29, wherein the V-shaped resilient member (35) further includes at least one resilient enhanced rib (353).

32. The safety syringe of claim 30, wherein the V-shaped resilient member (35) further includes at least one resilient enhanced rib (353).

33. The safety syringe of claim 27, wherein the wall member (34) is perpendicular to the moveable bifurcation (32) and includes a through hole (341), and wherein the latch (45) is inserted through the through hole (341) to be releasably secured thereto.

34. The safety syringe of claim 27, wherein the needle protection housing (40) further comprises a block member (43) at a rear end of the body (41), two side walls (42) on the body (41), at least one locking member (421) for needle (23) on an inner surface of at least one of the side walls (42), two opposite extensions (422), each extending forwardly from a front end of the side wall (42), and a space (44) defined by the extensions (422), the side walls (42), and the block member (43).
